(19) United States
(12) Patent Application Publication
(10) Pub. No.: US 2009/0100937 A1
(43) Pub. Date: Apr. 23, 2009

(54) Method of grading microfiber cleaning cloths

(75) Inventor: Bruno Niklaus, Cos Cob, CT (US)

Correspondence Address: GEORGE W. RAUCHFUSS;OHLANDT, GREELEY, RUGGIERO & PERLE, L.L.P., 10th FLOOR, ONE LANDMARK SQUARE, STAMFORD, CT, 06901-2682, US

(73) Assignee: UNGER MARKETING INTERNATIONAL, LLC.

(21) Appl. No.: 12/317,065

(22) Filed: Dec. 18, 2008

Related U.S. Application Data
(62) Division of application No. 11/418,048, filed on May 4, 2006.
(60) Provisional application No. 60/692,701, filed on Jun. 21, 2005.

(51) Int. Cl. G01N 33/00

(52) U.S. Cl. 73/760

(57) ABSTRACT

A method of grading a microfiber cleaning cloth is provided. The method includes determining a basis weight of the microfiber cleaning cloth; assigning the microfiber cleaning cloth a heavy-duty grade if the basis weight is greater than 400 g/m²; assigning the microfiber cleaning cloth a medium-duty grade if the basis weight is between about 300 to 400 g/m²; and assigning the microfiber cleaning cloth a light-duty grade if the basis weight is less than about 300 g/m².

### CROSS REFERENCE TO RELATED APPLICATIONS

This application is a divisional of U.S. application Ser. No. 11/418,048, which was filed on May 4, 2006, now pending, which in turn claims the benefit of U.S. Provisional Application Ser. No. 60/692,701, filed Jun. 21, 2005, the contents of both of which are incorporated by reference herein.

### BACKGROUND OF THE INVENTION

1. Field of the Invention

The present disclosure is related to microfiber cleaning cloths. More particularly, the present disclosure is related to a method of grading such microfiber cleaning cloths.

2. Description of Related Art

Microfiber cleaning cloths are knitted or weaved cloths made of polymer fibers. The polymer fibers are repeatedly split during the manufacturing process so that the cloth's cleaning surface includes a plurality of microfilaments, which are useful for trapping dirt, bacteria, and moisture. As such, microfiber cleaning cloths are fast becoming an essential cleaning tool for both industrial and domestic cleaning professionals, as well as every day consumers. For example, microfiber cleaning cloths can, when damp, be used to clean without chemicals, leaving a streak-free surface. When dry, the positively charged fibers of the cleaning cloths can attract and bond negatively charge particles, such as dust, to the cloth.

Microfiber cleaning cloths can be made with a variety of polymer materials, a variety of fiber sizes, and other attributes that effect one or more of the cost, the durability, and the suitability for particular cleaning tasks. This variety, while welcomed by the users, leads to much confusion during the selection of the appropriate microfiber cleaning cloth for a particular situation. For example, the type of microfiber cleaning cloth appropriate for use by a homeowner cleaning normal household objects differs substantially from the type of microfiber cleaning cloth appropriate for use by a professional cleaner cleaning industrial objects.

Accordingly, it has been determined by the present disclosure that there is a desire for a method of grading microfiber cleaning cloths.

### BRIEF SUMMARY OF THE INVENTION

It is an object of the present disclosure to provide a method of grading microfiber cloths.

It is another object of the present disclosure to provide a method of grading microfiber cloths that requires the determination of only four particular variables of the microfiber cloth.

It is yet a further object of the present disclosure to provide a method of grading microfiber cloths that requires the determination of only one variable of the microfiber cloth.

It is still a further object of the present disclosure to provide a method of grading a microfiber cloth that includes the use of both the grade of the microfiber cloth and a color of the cloth.

These and other objects and advantages of the present disclosure are provided by a method of grading a microfiber cleaning cloth, including: determining a basis weight of the microfiber cleaning cloth; assigning the microfiber cleaning cloth a heavy-duty grade if the basis weight is greater than 400 g/m²; assigning the microfiber cleaning cloth a medium-duty grade if the basis weight is between about 300 to 400 g/m²; and assigning the microfiber cleaning cloth a light-duty grade if the basis weight is less than about 300 g/m².

These and other objects and advantages of the present disclosure are also provided by a method of grading a microfiber cleaning cloth, including: determining a durability of the microfiber cleaning cloth; determining a washability of the microfiber cleaning cloth; determining an absorbency of the microfiber cleaning cloth; determining an abrasiveness of the microfiber cleaning cloth; and assigning a cloth grade to the microfiber cleaning cloth based only on the durability, washability, absorbency, and abrasiveness.

The above-described and other features and advantages of the present disclosure will be appreciated and understood by those skilled in the art from the following detailed description, drawings, and appended claims.

### BRIEF DESCRIPTION OF THE SEVERAL VIEWS OF THE DRAWINGS

FIG. 1 is a schematic depiction of a first exemplary embodiment of a method of grading microfiber cleaning cloths according to the present disclosure;

FIG. 2 is a schematic depiction of a second exemplary embodiment of a method of grading microfiber cleaning cloths according to the present disclosure; and

FIG. 3 is a schematic depiction of a third exemplary embodiment of a method of grading microfiber cleaning cloths according to the present disclosure.

### DETAILED DESCRIPTION OF THE INVENTION

The present disclosure has determined that microfiber cleaning cloths have a multitude of variables that can effect the performance and durability of the cloth. For example, the fiber thickness, fiber length, fiber finish, fiber abrasiveness, cloth absorbency, a variety of cloth weights, cloth style (e.g., terry cloth, suede finish, etc.), number of wear cycles, number of wash cycles, and others.

It has been determined by the present disclosure that the measurement and testing of these variables can be expensive, time consuming and difficult to correlate to actual cloth usage.

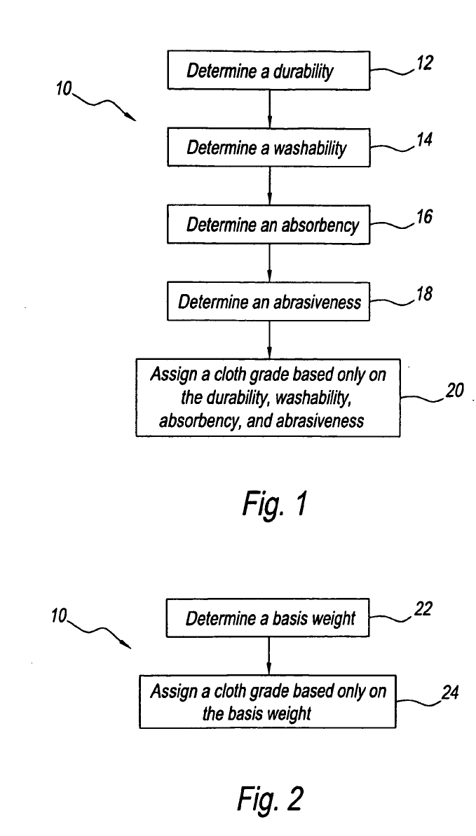

Referring now to the drawings and in particular to FIG. 1, a method 10 of grading microfiber cleaning cloths according to the present disclosure is shown.

It has been advantageously found that method 10 requires the determination of only four particular variables to successfully to grade microfiber cloths. Specifically, it has been found that determination of the number of wear cycles, the number of wash cycles, the water absorption, and abrasiveness of microfiber cloths can be used to grade the cloth.

For purposes of clarity, method 10 is described herein grading the microfiber cloth as one of a heavy-duty grade, a medium-duty grade, or a light-duty grade.

Method 10 includes four determination steps 12, 14, 16, 18 and a grade assigning step 20.

During determination step 12, the durability of the microfiber cloth is determined. The durability can be a measurement or estimation of a number of expected wear cycles of the cloth. For example, the number of expected wear cycles of the cloth can be measured as per ASTM test number D3886 or any other equivalent means of comparing wear data.

Determination step 14 determines the washability of the microfiber cloth using a measurement or estimation of a number of wash cycles of the cloth. For example, the number of wash cycles of the cloth can be measured by repeated washings of test samples and testing the samples at regular intervals for checking performance degradation. Wash cycle parameters must be kept consistent through out the length of the test and across different tests.

Determination step 16 determines the absorbency of the microfiber cloth using a measurement of the volume of liquid a predetermined size cloth can absorb. For example, the absorbency of the cloth can be measured by weighing the cloth before and after soaking, setting limits of water retention by the water stream coming out of the cloth when it is free standing or by allowing the cloth to drip for a standard time limit before taking the weight measurement.

Determination step 18 determines the abrasiveness of the microfiber cloth using a measurement of the coefficient of friction of the cloth. For example, the abrasiveness of the cloth can be measured as per ASTM test number F1677 or an equivalent means of comparing the friction of the cloth with a standard subtract. Several measurements with several subtracts can be made to add information to the test databank, for example, friction test with a specific cloth can be done on a dry vinyl floor tile, a dry glass window and a wet ceramic tile.

It should be recognized that the order of determination steps 12 through 18 is illustrated in FIG. 1 by way of example as a set of sequential steps in a particular order. Of course, it is contemplated by the present disclosure for the order of determination steps 12 through 18 to be any desired order and/or for determination steps 12 through 18 to occur simultaneous to one another. All these tests must also be made with new cloths as well as after the cloth have been laundered to have a clear understanding of the real cloth grade as the cloth performance usually differs form its original state after it has been laundered.

After steps 12 through 18 have been determined, the cloth grade can be assigned based only on the durability, washability, absorbency, and abrasiveness during assigning step 20.

Assigning step 20 can assign a heavy-duty grade if the durability is over about 4,000 wear cycles, the washability is over about 1,500 wash cycles, the absorbency is over about 1,700 ml/m², and the abrasiveness is over about 38. Assigning step 20 can assign a medium-duty grade if the durability is between about 2,000 and 4,000 wear cycles, the washability is between about 1,000 and 1,500 wash cycles, the absorbency is between about 1,400 ml/m² and 1,700 ml/m², the abrasiveness is between about 36 and 38. Assigning step 20 can assign a light-duty grade if the durability is between about 500 and 2,000 wear cycles, the washability is between about 500 and 1,000 wash cycles, the absorbency is between about 1,100 ml/m² and 1,400 ml/m², the abrasiveness is between about 34 and 36. In a preferred embodiment, assigning step 20 does not grade cloth with less performance than that of the light-duty grade.

The present disclosure has also surprisingly and advantageously found that the determination of only one variable can be successfully used to grade microfiber cloths. Specifically, it has been determined that the basis weight of the microfiber cloth alone, regardless of the values of the other variables discussed above, can be used to quickly and efficiently grade microfiber cloths.

Referring now to FIG. 2, method 10 is illustrated having one determination step 22 and a grade-assigning step 24. Determination step 22 determines the basis weight of the microfiber cloth. For example, determination step 22 can determine the basis weight of the microfiber cloth in grams per square meter (g/m²).

After steps 22 determines the basis weight, the cloth grade can be assigned based only on this weight during assigning step 24.

For example, assigning step 24 can assign a heavy-duty grade if the cloth has a basis weight of greater than 400 g/m², more particularly between about 400 to 450 g/m², with between about 420 to 435 g/m² being most preferred. Assigning step 24 can assign a medium-duty grade if the cloth has a basis weight of between about 300 to 400 g/m², with between about 320 to 350 g/m² being most preferred. Assigning step 24 can assign a light-duty grade if the cloth has a basis weight of less than about 300 g/m², more particularly between about 250 to 290 g/m², with between about 265 to 275 g/m² being most preferred.

Advantageously, it has been determined by the present disclosure that the selection and use of microfiber cloths can be simplified using both the grade of the microfiber cloth and a color of the cloth.

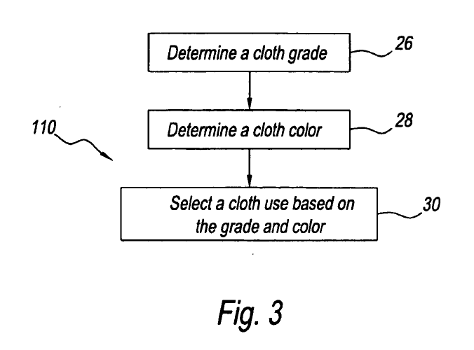

Referring now to FIG. 3, a method 110 of using the cloth grade obtained using method 10 is shown. Method 110 includes a cloth grading step 26, a cloth color determination step 28, and a cloth selection step 30. Cloth selection step 30 is based upon the results obtained by cloth grading step 26 and color determination step 28.

Cloth-grading step 26 can find use with method 10 described herein above. Namely, cloth-grading step 26 can determine whether the microfiber cloth is a heavy-duty cloth, a medium-duty cloth, or a light-duty cloth based upon the four variables of FIG. 1 or the single variable of FIG. 2.

In color-determination step 28, the color of the cloth can be determined. For example, the cloth can be provided in one of a plurality of colors. In this manner, the cloth selected for one particular area to be cleaned (i.e., a bathroom) can be used exclusively for that area without being used in another particular area (i.e., kitchen). For example, the cloth can be provided in a red color for use in a bathroom, a green color for use in a kitchen, and a blue color for use in non-specific areas.

Cloth selection step 30 allows the user to select the appropriate cloth based upon the results obtained by cloth grading step 26 and color determination step 28. Here, the color determination step 28 can prevent cross contamination among areas to be cleaned while cloth grading step 26 assists the user to select the most appropriate cloth for the area being cleaned.

It should also be noted that the terms “first”, “second”, “third”, “upper”, “lower”, and the like may be used herein to modify various elements. These modifiers do not imply a spatial, sequential, or hierarchical order to the modified elements unless specifically stated.

While the present disclosure has been described with reference to one or more exemplary embodiments, it will be understood by those skilled in the art that various changes may be made and equivalents may be substituted for elements thereof without departing from the scope of the present disclosure. In addition, many modifications may be made to adapt a particular situation or material to the teachings of the disclosure without departing from the scope thereof. Therefore, it is intended that the present disclosure not be limited to the particular embodiment(s) disclosed as the best mode contemplated, but that the disclosure will include all embodiments falling within the scope of the appended claims.

What is claimed is:

1. A method of grading a microfiber cleaning cloth, comprising:
determining a durability of the microfiber cleaning cloth;
determining a washability of the microfiber cleaning cloth;
determining an absorbency of the microfiber cleaning cloth;
determining an abrasiveness of the microfiber cleaning cloth; and
assigning a cloth grade to the microfiber cleaning cloth based only on said durability, washability, absorbency, and abrasiveness.

2. The method as in claim 1, wherein assigning said cloth grade comprises:
assigning a heavy-duty grade if said durability is over about 4,000 number of wear cycles, said washability is over about 1,500 number of wash cycles, said absorbency is over about 1,700 ml/m², and said abrasiveness is over about 38;
assigning a medium-duty grade if said durability is between about 2,000 and 4,000 wear cycles, said washability is between about 1,000 and 1,500 of wash cycles, said absorbency is between about 1,400 ml/m² and 1,700 ml/m², and said abrasiveness is between about 36 and 38; and
assigning a light-duty grade if said durability is between about 500 and 2,000 wear cycles, said washability is between about 500 and 1,000 wash cycles, said absorbency is between about 1,100 ml/m² and 1,400 ml/m², and said abrasiveness is between about 34 and 36.

3. The method as in claim 2, further comprising:
assigning all of the microfiber cleaning cloths having said heavy-duty grade a first color;
assigning all of the microfiber cleaning cloths having said medium-duty grade a second color;
assigning all of the microfiber cleaning cloths having said light-duty grade a third color;
selecting which the microfiber cleaning cloths to use for a particular job based on said first, second or third color.

4. A method of grading a microfiber cleaning cloth, comprising:
determining a first absorbency, a first abrasiveness, and first durability of the microfiber cleaning cloth before determining a washability of the microfiber cleaning cloth;
determining said washability of the microfiber cleaning cloth;
determining a second absorbency, a second abrasiveness, and a second durability of the microfiber cleaning cloth after determining said washability; and
assigning a cloth grade to the microfiber cleaning cloth based only on said first and second durability, said first and second absorbency, said first and second abrasiveness, and said washability.

5. The method as in claim 4, wherein assigning said cloth grade comprises assigning one of a heavy-duty grade, a medium-duty grade, and a light-duty grade.

6. The method as in claim 4, wherein assigning said cloth grade comprises assigning a heavy-duty grade if said first and second durability are over about 4,000 number of wear cycles, said washability is over about 1,500 number of wash cycles, said first and second absorbency are over about 1,700 ml/m², and said first and second abrasiveness are over about 38.

7. The method as in claim 4, wherein assigning said cloth grade comprises assigning a medium-duty grade if said first and second durability are between about 2,000 and 4,000 wear cycles, said washability is between about 1,000 and 1,500 of wash cycles, said first and second absorbency are between about 1,400 ml/m² and 1,700 ml/m², and said first and second abrasiveness are between about 36 and 38.

8. The method as in claim 4, wherein assigning said cloth grade comprises assigning a light-duty grade if said first and second durability are between about 500 and 2,000 wear cycles, said washability is between about 500 and 1,000 wash cycles, said first and second absorbency are between about 1,100 ml/m² and 1,400 ml/m², and said first and second abrasiveness are between about 34 and 36.

9. The method as in claim 4, further comprising:
assigning all of the microfiber cleaning cloths having said heavy-duty grade a first color;
assigning all of the microfiber cleaning cloths having said medium-duty grade a second color;
assigning all of the microfiber cleaning cloths having said light-duty grade a third color;
selecting which of the microfiber cleaning cloths to use for a particular job based on said first, second or third color.
